(19) United States
(12) Patent Application Publication
(10) Pub. No.: US 2006/0159762 A1
(43) Pub. Date: Jul. 20, 2006

(54) Stable pharmaceutical composition comprising an active substance in the form of solid solution

(76) Inventors: Tijana Stanic Ljubin, Ljubljana (SI); Judita Sirca, Ljubljana (SI)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/317,769

(22) Filed: Dec. 23, 2005

(30) Foreign Application Priority Data
Dec. 24, 2004 (SI) P200400351

(51) Int. Cl. A61K 31/4439; A61K 9/24

(52) U.S. Cl. 424/472; 514/338

(57) ABSTRACT

The present invention relates to a novel pharmaceutical comprising an active substance in the form of solid solution. The stability of active substance in the pharmaceutical composition is significantly improved relative to the stability of non-formulated active substance.

### FIELD OF THE INVENTION

The present invention belongs to the field of pharmaceutical technology and relates to a novel pharmaceutical composition comprising an active substance in the form of solid solution. The stability of the active substance in the pharmaceutical composition is significantly improved relative to the stability of the active substance itself.

More particularly, the invention relates to a stable pharmaceutical composition comprising an active substance that is unstable in acidic medium, unstable when stored in the presence of water and at the same time sensitive to heating said active substance being in the form of solid solution. Further, the invention relates to a method of stabilization of said active substance by transforming said active substance into the form of solid solution and to a process of preparation of the stable pharmaceutical composition comprising the active substance in the form of solid solution.

### BACKGROUND OF THE INVENTION

There is a constant need for developing a stable pharmaceutical composition, wherein a good stability of an active substance, that is unstable in acidic medium, unstable when stored in presence of water and at the same time sensitive to heating, is achieved.

The use of solid dispersions and solid solutions is known principle in pharmaceutical industry for reducing particle size to a molecular level, in order to enhance oral bioavailability of poorly water soluble drugs. This concept was introduced in Chem. Pharm. Bull., 9 (1961), 866-872 by Sekiguchi and Obi and later in J. Pharm. Sci., 60 (1971a) 1281-1302 by Chiou, W. U and Riegelman, S. The use of solid dispersion in the preparation of pharmaceutical composition for enhancing oral bioavailability is also disclosed in Eur. J. of Pharm. and Biopharm., 50 (1), (2000), 47-60 by Leuner C. and Dressman J.

A pharmaceutical formulation for oral administration comprising pure solid state salts of esomeprazole is disclosed in U.S. Pat. No. 5,714,504. The described solid state salt of esomeprazole of Na⁺, Mg²⁺, Li⁺, K⁺, Ca²⁺ or N⁺(R)₄, is optically pure and substantially crystalline.

WO 96/01623 discloses an oral pharmaceutical multiple unit tableted dosage form comprising tablet excipients and individually enteric coating layered units of a core material containing active substance in the form of omeprazole or one of its single enantiomers or an alkaline salt of omeprazole or one of its single enantiomers, optionally the active substance is mixed with alkaline compounds and pharmaceutically acceptable excipients, the core material is covered with one or more layer(s), of which at least one is an enteric coating layer, characterised in that the enteric coating layer comprises a plasticizer in the amount of 20-50% by weight of the enteric coating layer polymer and that the enteric coating layer has mechanical properties such that the compression of the individual units mixed with the tablet excipients into the multiple unit tableted dosage form does not significantly affect the acid resistance of the individually enteric coating layered units.

Thus in hitherto known patent and related literature no reference discloses the problem of poor stability of active substances that are unstable in acidic medium, unstable when stored in presence of water and at the same time sensitive to heating, being solved by transforming said active substance into the form of solid solution.

### SUMMARY OF THE INVENTION

In a first aspect, the present invention relates to a stable pharmaceutical composition comprising an active substance that is unstable in acidic medium, unstable when stored in the presence of water and at the same time sensitive to heating, wherein said active substance is stabilized by being transformed into the form of solid solution.

The stable pharmaceutical composition according to the present invention comprises a core material containing an inert core and solid solution layer, optionally one or more subcoatings and an enteric coating.

In a second aspect, the present invention relates to a method of stabilization of an active substance that is unstable in acidic medium, unstable when stored in the presence of water and at the same time sensitive to heating, in the pharmaceutical composition, by transforming said active substance into the form of solid solution.

In a third aspect, the present invention relates to a process for the preparation of the stable pharmaceutical composition according to the present invention comprising the steps of a) providing a core material, comprising preparing and applying of a layer containing an active substance in the form of solid solution on the surface of the inert core, b) optionally applying one or more subcoatings on the core material, and c) applying enteric coating.

### DETAILED DESCRIPTION OF THE INVENTION

Transformation of an active substance into the form of solid solution is a well known technology used hitherto to increase the solubility of active substance. However, we have surprisingly found that by transforming an active substance that is unstable in acidic medium, unstable when stored in presence of water and at the same time sensitive to heating, into the form of solid solution a stable pharmaceutical composition containing said active substance has been obtained. The advantage of the stable pharmaceutical composition according to the present invention is its stability being significantly improved relative to the stability of non-formulated active substance, which means that the amount of degradation products of the active substance in the pharmaceutical composition according to the present invention is minimized in comparison to the amount of the degradation products of non-formulated active substance, when stored under the same conditions as the stable pharmaceutical composition according to the present invention. In J. Pharm. Sci., 60 (1971a) 1281-1302 by Chiou, W. U and Riegelman, S. the term solid dispersion is defined as “a dispersions of one or more active ingredients in an inert carrier or matrix at solid state prepared by the melting (fusion), solvent or melting-solvent methods”. Dispersions obtained through the fusion process are often called melts, and those obtained by the solvent method are frequently referred to as coprecipitates or coevaporates. Chiou and Riegelman classified solid dispersions into the following six representative types: simple eutectic mixtures, solid solutions, glass solutions and glass suspensions, amorphous precipitations in a crystalline carrier, compound or complex formation, and combinations thereof.

In a solid solution the two components form a homogeneous one-phase system. The particle size of the drug in the solid solution is reduced to its molecular size. Many techniques have been used to characterize the physical nature of solid dispersions, including thermal analysis (as for example cooling curve, thaw melt, thermomicroscopy and DTA methods), x-ray diffraction, microscopic, spectroscopic, dissolution rate, and thermodynamic methods. Usually, a combination of two or more methods is required to obtain a complete picture of the solid dispersion system.

Therefore, the first object of the present invention is to provide a stable pharmaceutical composition comprising an active substance that is unstable in acidic medium, unstable when stored in the presence of water and at the same time sensitive to heating, wherein said active substance is stabilized by being transformed into the form of solid solution.

The stable pharmaceutical composition according to the present invention comprises:

a) a core material comprising an inert core and a solid solution layer.

b) optionally one or more subcoatings,

c) an enteric coating.

A suitable inert core present in the core material of the stable pharmaceutical composition according to the present invention may be, for example, a non-pareil bead, a crystal, a granule, a pellet, a spherule, a micro tablet or a tablet. A preferred inert core according to the present invention is a non-pareil bead, for example a non-pareil bead made of microcrystalline cellulose, sucrose, starch or any combinations thereof, and preferably a non-pareil bead made of sucrose and starch.

A suitable solid solution layer comprises the solid solution of an active substance in a polymer carrier and other pharmaceutical excipients necessary for film coating.

As an active substance present in the solid solution layer of the core material of the stable pharmaceutical composition according to the present invention various active substances that are unstable in acidic medium, unstable when stored in presence of water and at the same time sensitive to heating, can be used. Said active substances can be selected from the group consisting of analgesics, anticonvulsants, antiparkinsonics, anaesthetics, antibiotics, antimalarial agents, antihypertensives, antihistaminics, anti-obesity agents, serum lipid reducing agents, antipyretics, alpha-blockers, alpha-adrenergic agonists, bactericides, bronchial dilators, beta-adrenergic stimulants, beta-adrenergic blockers, enzymes, contraceptives, cardiovascular active substances, calcium channel inhibitors, proton pump inhibitors, diuretics, hypnotics, hormones, hyperglycemics, hypoglycemics, muscle relaxants and contractors, parasympathomimetics, sedatives, sympathomimetics, tranquillizers, antimigraine agents, vitamins and any combinations thereof.

The stable pharmaceutical composition according to the present invention is especially suitable for various benzimidazole derivatives, acting as proton pump inhibitors, such as omeprazole, lansoprazole, timoprazole, rabeprazole, pantoprazole, leminoprazole, pariprazole, their pharmaceutically acceptable salts, their enantiomers and pharmaceutically acceptable salts of their enantiomers, preferably magnesium salt of esomeprazole.

Suitable polymer carrier present in the solid solution layer of the core material of the stable pharmaceutical composition according to the present invention includes polyvinylpyrrolidone of different grades, cellulose derivatives, such as for example hydroxypropyl methhylcellulose acetate succinate, hydroxypropyl methylcellulose, hydroxypropyl methylcellulose phthalate, ethylcellulose, polymethacrylates, polyethylene glycols and any combinations thereof. Preferably polyvinylpyrrolidone of different grades and any combinations thereof might be used as the polymer carrier.

The weight ratio between the active substance and the polymer carrier present in the solid solution layer of the core material of the stable pharmaceutical composition according to the present invention has to be predefined to result in forming a solid solution. The preferred weight ratio is from 1:1 to 1:6, in particular from 1:1 to 1:3. The predefined weight ratio may be determined by using X-ray diffraction analysis, differential scanning calorimetry (DSC) or Raman microscopy. Before mentioned methods have proven that the core material of the pharmaceutical composition according to the present invention really comprises a solid solution of the active substance in the polymer carrier and not their physical mixture.

The solid solution layer of the core material of the stable pharmaceutical composition according to the present invention advantageously comprises pharmaceutically acceptable excipients necessary for film coating, such as for example, one or more plasticizing agents, for example dibutyl sebacate, triethyl citrate or diethyl phthalate, one or more surface active agents, for example polysorbate or sodium lauryl sulfate and optionally anti-tacking agents, for example talcum, glyceryl monostearate or magnesium stearate.

Before applying the enteric coating onto the core material of the stable pharmaceutical composition according to the present invention, optionally one or more subcoatings may be applied onto said core material.I. The optional subcoating comprises at least one film forming polymer such as for example cellulose ethers, as for example hydroxypropyl methylcellulose, hydroxypropylcellulose, methylcellulose, sodium carboxymethylcellulose, ethylcellulose, polyvinylpyrrolidones, vinyl pyrrolidone/vinyl acetate copolymer, polymethacrylates and other common pharmaceutically acceptable excipients that are used in the preparation of film coatings, such as plasticizers, as for example polyethylene glycols of different molecular weight, cetyl alcohol, olive oil, castor oil, monoglycerides, diethyl phthalate, triethyl citrate, dibutyl sebacate, anti-tacking agent, as for example talcum, glyceryl monostearate, magnesium stearate, surface active agents, as for example polysorbate, sodium lauryl sulfate. The amount of applied one or more subcoatings is from 0% to 20%, preferably from 5% to 15%, relative to the total weight of the core material and the subcoating.

The enteric coating of the stable pharmaceutical composition according to the present invention comprises at least one polymer soluble at higher pH values, as for example higher than about pH 5.0 such as copolymers of methacrylic acid, ethyl cellulose, shellac, esters of hydroxyalkylcellulose, preferably hydroxypropyl methylcellulose phthalate, one or more plasticizers, such as polyethylene glycols of different molecular weight, cetyl alcohol, olive oil, castor oil, monoglycerides, diethyl phthalate, triethyl citrate, preferably dibutyl sebacate and other common pharmaceutically acceptable excipients that are used in the preparation of the enteric coating, such as an anti-tacking agent, as for example talcum, surface active agents, as for example polysorbate, pigments etc. The amount of applied enteric coating is, for example, from 10% to 50%, preferably from 20% to 35% each relative to the total weight of the pharmaceutical composition.

A further object of the present invention is a method of stabilizing an active substance, that is unstable in acidic medium, unstable when stored in the presence of water and at the same time sensitive to heating, in the pharmaceutical composition, by transforming it into the form of a solid solution. The stability of said active substance in the pharmaceutical composition according to the present invention, wherein the active substance is in the form of a solid solution, is significantly improved. The amount of the degradation products of the active substance in the pharmaceutical composition according to the present invention is significantly less in comparison to the amount of the degradation products of the non-formulated active substance, when stored under the same conditions as the stable pharmaceutical composition according to the present invention.

It is believed that the mechanism of stability improvement is steric fixation and/or isolation of individual active substance molecules in the matrix of polymer carrier, so that the molecules of the active substance fixated and/or isolated by this mechanism are protected from influences of acidic medium, presence of water and heating. Therefore, the molecules are significantly less susceptible to chemical degradation processes in comparison to non-formulated molecules.

A further object of the present invention is a process for the preparation of a stable pharmaceutical composition according to the present invention, comprising an active substance in the form of solid solution.

The process of the preparation of a stable pharmaceutical composition according to the present invention comprises:

a) providing a core material, that comprises preparing and applying of a layer containing an active substance in the form of a solid solution onto the surface of an inert core,

b) optionally applying one or more subcoatings onto the core material,

c) applying an enteric coating onto the core material.

The core material is prepared by applying a solid solution layer onto an inert core. The solid solution layer is prepared, for example, by the solvent method. To this end the active substance and a polymer carrier are dissolved in one or more pharmaceutically acceptable organic solvents, preferably selected from the group of ethanol and acetone, more preferably ethanol. Other pharmaceutical excipients necessary for film coating may be dissolved or dispersed in the same solvent/s. The solvent is then evaporated, for example, by spraying the obtained dispersion onto inert cores by using a suitable coating technique, for example a fluid-bed system or a conventional coating pan. During the solvent evaporation phase, the solid solution of the active substance in the polymer carrier is formed as a layer on the surface of the inert cores.

The obtained core material can be further dried by using a suitable drying technique, for example a fluid-bed system, a conventional coating pan, a tray or a truck-drier.

An enteric coating may be applied onto the core material by using a suitable coating technique, for example a fluid-bed system or a conventional coating pan.

Between the core material and the enteric coating, one or more optional subcoatings may be applied, by using a suitable coating technique, for example a fluid-bed system or a conventional coating pan.

The further processing of the stable pharmaceutical composition according to the present invention depends on the type of inert cores used in the core material.

When inert tablets are used as inert cores of the core material, the stable pharmaceutical composition according to the present invention might represent a final dosage form without further processing.

When non-pareil beads, crystals, granules, spherules or micro tablets are used as inert cores, the stable pharmaceutical composition according to the present invention can be filled into sachettes or capsules; preferred capsules are hard capsules, in particular capsules made of hydroxypropylmetylcellulose.

When non-pareil beads, crystals, granules, spherules and micro tablets are used as inert cores, the stable pharmaceutical composition according to the present invention can also be compacted into tablets together with pharmaceutically acceptable excipients such as tablet fillers, binders, disintegrating agents, glidants, lubricants etc. The stable pharmaceutical composition according to the present invention can be compacted into tablets after being mixed with the simple powder mixture of said excipients, or with agglomerations of said excipients, such as granules, spherules, beads, micropellets, pellets, etc., which can be prepared, for example, by wet granulation, hot melt pelletization, thermoplastic pelletization, extrusion and spheronisation, spray drying, freeze drying, or any other common method for obtaining said agglomerations.

Tablets obtained with compacting the stable pharmaceutical composition according to the present invention together with pharmaceutically acceptable excipients could be further coated with a film coating, comprising film forming polimers, such as cellulose derivatives, polyvinylpyrrolidones, polymethacrylates etc, or any combination thereof. Preferred film coatings comprise polymers soluble in organic solvents, for example hydroxypropylcellulose, and other common pharmaceutically acceptable excipients that are used in the preparation of film coatings, such as plasticizers, anti-tacking agent, surface active agents, pigments, etc.

The stable pharmaceutical composition according to the present invention is especially suitable for various benzimidazole derivatives acting as proton pump inhibitors that are used for inhibiting gastric secretion in mammals in man, preferably omeprazole, its salts, its single enantiomers and its single enantiomers salts, preferably esomeprazole magnesium. The stable pharmaceutical composition according to the present invention might contain from 20 mg and 100 mg dose of esomeprazole.

The stable pharmaceutical composition according to the present invention comprising a benzimidazole derivative may be used for the treatment of gastric acid-related diseases and gastrointestinal inflammatory diseases in mammals and man, such as gastric ulcer, duodenal ulcer, reflux esophagitis, and gastritis, for treatment of other gastrointestinal disorders where gastric antisecretory effect is desirable e.g. in patients on NSAID therapy, in patients with gastrinomas, and in patients with acute upper gastrointestinal bleeding. It may also be used in patients in intensive care situations, and pre- and postoperatively to prevent acid aspiration and stress ulceration. The stable pharmaceutical composition according to the present invention comprising benzimidazole derivatives may also be used for treatment or prophylaxis of inflammatory conditions in mammals, including man, especially those involving lysozymal enzymes, such as rheumatoid arthritis and gout. The stable pharmaceutical composition according to the present invention comprising benzimidazole derivatives may also be useful in the treatment of psoriasis as well as in the treatment of Helicobacter infections.

The invention is illustrated by the following examples, which in no way limit the scope thereof.

### EXAMPLE 1

CORE MATERIAL (weight 174.000 mg)

| | | | |
|---|---|---|---|
| Non-pareil beads | 100.300 | mg | |
| Esomeprazole magnesium | 20.000 | mg | Active substance |
| Polyvinylpyrrolidone K-30 | 50.000 | mg | Polymer carrier |
| Sodium lauryl sulfate | 1.850 | mg | Surface active agent |
| Talcum | 1.850 | mg | Anti-tacking agent |
| Ethanol | 417.700 | mg | Organic solvent |

Method of Preparation of Core Material

Polyvinylpyrrolidone K-30 and esomeprazole magnesium are dissolved in ethanol, then sodium lauryl sulfate is dissolved and talcum is dispersed in the obtained solution. The obtained dispersion is bottom sprayed onto non-pareil beads in fluid-bed device. During this process ethanol evaporates and solid solution of esomeprazole magnesium in polyvinylpyrrolidone is formed as a layer on the surface of non-pareil beads.

The obtained core material is dried in fluid-bed device.

ENTERIC COATING (25% application,
coating weight 58.000 mg)

| | | | |
|---|---|---|---|
| Hydroxypropyl methylcellulose | 56.376 | mg | Enteric polymer |
| phthalate HP-50 | | | |
| Dibutyl sebacate | 1.044 | mg | Plasticizer |
| Talcum | 0.580 | mg | Anti tacking agent |
| Ethanol | 446.154 | mg | Solvent |
| Acetone | 446.154 | mg | Solvent |

Method of Preparation of Enteric Coating

Hydroxypropyl methylcellulose phthalate HP-50 and dibutyl sebacate are dissolved in the mixture of ethanol and acetone. Talcum is suspended in obtained solution. The obtained dispersion is bottom sprayed in fluid-bed device onto the core material.

### EXAMPLE 2

CORE MATERIAL (weight 150.000 mg)

| | | | |
|---|---|---|---|
| Non-pareil beads | 72.600 | mg | |
| Esomeprazole magnesium | 20.000 | mg | Active substance |
| Polyvinylpyrrolidone K-25 | 50.000 | mg | Polymer carrier |
| Diethyl phthalate | 3.700 | mg | Plasticizer |
| Polysorbate | 1.850 | mg | Surface active agent |
| Talcum | 1.850 | mg | Anti-tacking agent |
| Ethanol | 696.600 | mg | Organic solvent |

Method of Preparation of Core Material

Polyvinylpyrrolidone K-25 and esomeprazole magnesium are dissolved in ethanol, then polysorbate and diethyl phthalate are dissolved and talcum is dispersed in the obtained solution. The obtained dispersion is bottom sprayed onto non-pareil beads in fluid-bed device. During this process ethanol evaporates and solid solution of esomeprazole magnesium in polyvinylpyrrolidone is formed as a layer on the surface of non-pareil beads. The obtained core material is dried in fluid-bed device.

SUBCOATING (12% application, coating weight 20.445 mg)

| | | | |
|---|---|---|---|
| Polyvinylpyrrolidone K-25 | 14.500 | mg | Polymer carrier |
| Sodium lauryl sulfate | 0.537 | mg | Surface active agent |
| Diethyl phthalate | 1.073 | mg | Plasticizer |
| Talcum | 4.345 | mg | Anti-tacking agent |
| Ethanol | 184.091 | mg | Organic solvent |

Method of Preparation of Subcoating

Sodium lauryl sulfate, polyvinylpyrrolidone and diethyl phthalate are dissolved in ethanol. Talcum is suspended in the obtained solution. The obtained dispersion is bottom sprayed in fluid-bed device onto the core material.

ENTERIC COATING (25% application,
coating weight 56.818 mg)

| | | | |
|---|---|---|---|
| Hydroxypropyl methylcellulose | 54.659 | mg | Enteric polymer |
| phthalate HP-50 | | | |
| Dibutyl sebacate | 1.023 | mg | Plasticizer |
| Talcum | 1.136 | mg | Anti-tacking agent |
| Ethanol | 437.063 | mg | Organic solvent |
| Acetone | 437.063 | mg | Organic solvent |

Method of Preparation of Enteric Coating

Hydroxypropyl methylcellulose phthalate HP-50 and dibutyl sebacate are dissolved in the mixture of ethanol and acetone. Talcum is suspended in obtained solution. The obtained dispersion is bottom sprayed in fluid-bed device onto the core material covered with subcoating.

### EXAMPLE 3

CORE MATERIAL (weight 150.000 mg)

| | | | |
|---|---|---|---|
| Non-pareil beads | 72.600 | mg | |
| Omeprazole | 20.000 | mg | Active substance |
| Polyvinylpyrrolidone K-25 | 50.000 | mg | Polymer carrier |
| Diethyl phthalate | 3.700 | mg | Plasticizer |
| Polysorbate | 1.850 | mg | Surface active agent |
| Talcum | 1.850 | mg | Anti-tacking agent |
| Ethanol | 696.600 | mg | Organic solvent |

Method of Preparation of a Core Material

Polyvinylpyrrolidone K-25 and omeprazole are dissolved in ethanol, then polysorbate and diethyl phthalate are dissolved and talcum is dispersed in the obtained solution. The obtained dispersion is bottom sprayed onto non-pareil beads in fluid-bed device. During this process ethanol evaporates and solid solution of omeprazole in polyvinylpyrrolidone is formed as a layer on the surface of non-pareil beads.

The obtained core material is dried in fluid-bed device.

SUBCOATING (12% application, coating weight 20.445 mg)

| | | | |
|---|---|---|---|
| Polyvinylpyrrolidone K-25 | 14.500 | mg | Polymer carrier |
| Sodium lauryl sulfate | 0.537 | mg | Surface active agent |
| Diethyl phthalate | 1.073 | mg | Plasticizer |
| Talcum | 4.345 | mg | Anti-tacking agent |
| Ethanol | 184.091 | mg | Organic solvent |

Method of Preparation of Subcoating

Sodium lauryl sulfate, polyvinylpyrrolidone and diethyl phthalate are dissolved in ethanol. Talcum is suspended in the obtained solution. The obtained dispersion is bottom sprayed in fluid-bed device onto the core material.

ENTERIC COATING (25% application,
coating weight 56.818 mg)

| | | | |
|---|---|---|---|
| Hydroxypropyl methylcellulose | 54.659 | mg | Enteric polymer |
| phthalate HP-50 | | | |
| Dibutyl sebacate | 1.023 | mg | Plasticizer |
| Talcum | 1.136 | mg | Anti-tacking agent |
| Ethanol | 437.063 | mg | Organic solvent |
| Acetone | 437.063 | mg | Organic solvent |

Method of Preparation of Enteric Coating

Hydroxypropyl methylcellulose phthalate HP-50 and dibutyl sebacate are dissolved in the mixture of ethanol and acetone. Talcum is suspended in obtained solution. The obtained dispersion is bottom sprayed in fluid-bed device onto the core material covered with subcoating.

### EXAMPLE 4

CORE MATERIAL (weight 150.000 mg)

| | | | |
|---|---|---|---|
| Non-pareil beads | 72.600 | mg | |
| Lansoprazole | 20.000 | mg | Active substance |
| Polyvinylpyrrolidone K-25 | 50.000 | mg | Polymer carrier |
| Diethyl phthalate | 3.700 | mg | Plasticizer |
| Polysorbate | 1.850 | mg | Surface active agent |
| Talcum | 1.850 | mg | Anti-tacking agent |
| Ethanol | 696.600 | mg | Organic solvent |

Method of Preparation of a Core Material

Polyvinylpyrrolidone K-25 and lansoprazole are dissolved in ethanol, then polysorbate and diethyl phthalate are dissolved and talcum is dispersed in the obtained solution. The obtained dispersion is bottom sprayed onto non-pareil beads in fluid-bed device. During this process ethanol evaporates and solid solution of lansoprazole in polyvinylpyrrolidone is formed as a layer on the surface of non-pareil beads.

The obtained core material is dried in fluid-bed device.

| | | | |
|---|---|---|---|
| Ethanol | 184.091 | mg | Organic solvent |

SUBCOATING (12% application, coating weight 20.445 mg)

| | | | |
|---|---|---|---|
| Polyvinylpyrrolidone K-25 | 14.500 | mg | Polymer carrier |
| Sodium lauryl sulfate | 0.537 | mg | Surface active agent |
| Diethyl phthalate | 1.073 | mg | Plasticizer |
| Talcum | 4.345 | mg | Anti-tacking agent |

Method of Preparation of Subcoating

Sodium lauryl sulfate, polyvinylpyrrolidone and diethyl phthalate are dissolved in ethanol. Talcum is suspended in the obtained solution. The obtained dispersion is bottom sprayed in fluid-bed device onto the core material.

ENTERIC COATING (25% application,
coating weight 56.818 mg)

| | | | |
|---|---|---|---|
| Hydroxypropyl methylcellulose | 54.659 | mg | Enteric polymer |
| phthalate HP-50 | | | |
| Dibutyl sebacate | 1.023 | mg | Plasticizer |
| Talcum | 1.136 | mg | Anti-tacking agent |
| Ethanol | 437.063 | mg | Organic solvent |
| Acetone | 437.063 | mg | Organic solvent |

Method of Preparation of Enteric Coating

Hydroxypropyl methylcellulose phthalate HP-50 and dibutyl sebacate are dissolved in the mixture of ethanol and acetone. Talcum is suspended in obtained solution. The obtained dispersion is bottom sprayed in fluid-bed device onto the core material covered with subcoating.

### EXAMPLE 5

CORE MATERIAL (weight 138.000 mg)

| | | | |
|---|---|---|---|
| Non-pareil beads | 71.340 | mg | |
| Esomeprazole magnesium | 22.200 | mg | Active substance |
| Polyvinylpyrrolidone K-25 | 39.960 | mg | Polymer carrier |
| Dibutyl sebacate | 3.000 | mg | Plasticizer |
| Polysorbate | 1.500 | mg | Surface active agent |
| Ethanol | 504.197 | mg | Organic solvent |

Method of Preparation of a Core Material

Polysorbate, polyvinylpyrrolidone K-25, dibutyl sebacate, and esomeprazole magnesium are dissolved in ethanol. The obtained solution is bottom sprayed onto non-pareil beads in fluid-bed device. During this process ethanol evaporates and solid solution of esomeprazole magnesium in polyvinylpyrrolidone is formed as a layer on the surface of non-pareil beads. The obtained core material is dried in fluid-bed device.

SUBCOATING (8% application, coating weight 12.000 mg)

| | | | |
|---|---|---|---|
| Polyvinylpyrrolidone K-25 | 4.320 | mg | Film forming polymer |
| Dibutyl sebacate | 0.216 | mg | Plasticizer |
| Talcum | 7.464 | mg | Anti-tacking agent |
| Ethanol | 108.000 | mg | Organic solvent |

Method of Preparation of Subcoating

Polyvinylpyrrolidone and dibutyl sebacate are dissolved in ethanol. Talcum is suspended in the obtained solution. The obtained dispersion is bottom sprayed in fluid-bed device onto the core material.

ENTERIC COATING (25% application,
coating weight 50.000 mg)

| | | | |
|---|---|---|---|
| Hydroxypropyl methylcellulose | 31.000 | mg | Enteric polymer |
| phthalate HP-55 | | | |
| Dibutyl sebacate | 1.500 | mg | Plasticizer |
| Talcum | 16.000 | mg | Anti-tacking agent |
| Glyceryl monostearate | 1.500 | mg | Anti-tacking agent |
| Ethanol | 332.143 | mg | Organic solvent |
| Acetone | 332.143 | mg | Organic solvent |

Method of Preparation of Enteric Coating

Hydroxypropyl methylcellulose phthalate HP-55 and dibutyl sebacate are dissolved in the mixture of ethanol and acetone. Talcum and glyceryl monostearate are suspended in obtained solution. The dispersion is bottom sprayed in fluid-bed device onto the core material covered with subcoating.

The stable pharmaceutical composition disclosed in this example is filled into hydroxypropyl metylcellulose hard capsules.

### EXAMPLE 6

CORE MATERIAL (weight 276.000 mg)

| | | | |
|---|---|---|---|
| Non-pareil beads | 142.680 | mg | |
| Esomeprazole magnesium | 44.400 | mg | Active substance |
| Polyvinylpyrrolidone K-25 | 79.920 | mg | Polymer carrier |
| Dibutyl sebacate | 6.000 | mg | Plasticizer |
| Polysorbate | 3.000 | mg | Surface active agent |
| Ethanol | 1008.394 | mg | Organic solvent |

Method of Preparation of a Core Material

Polysorbate, polyvinylpyrrolidone K-25, dibutyl sebacate, and esomeprazole magnesium are dissolved in ethanol. The obtained solution is bottom sprayed onto non-pareil beads in fluid-bed device. During this process ethanol evaporates and solid solution of esomeprazole magnesium in polyvinylpyrrolidone is formed as a layer on the surface of non-pareil beads. The obtained core material is dried in fluid-bed device.

SUBCOATING (8% application, coating weight 24.000 mg)

| | | | |
|---|---|---|---|
| Polyvinylpyrrolidone K-25 | 8.640 | mg | Film forming polymer |
| Dibutyl sebacate | 0.432 | mg | Plasticizer |
| Talcum | 14.928 | mg | Anti-tacking agent |
| Ethanol | 216.000 | mg | Organic solvent |

Method of Preparation of Subcoating

Polyvinylpyrrolidone and dibutyl sebacate are dissolved in ethanol. Talcum is suspended in the obtained solution. The obtained dispersion is bottom sprayed in fluid-bed device onto the core material.

ENTERIC COATING (25% application,
coating weight 100.000 mg)

| | | | |
|---|---|---|---|
| Hydroxypropyl methylcellulose | 62.000 | mg | Enteric polymer |
| phthalate HP-55 | | | |
| Dibutyl sebacate | 3.000 | mg | Plasticizer |
| Talcum | 32.000 | mg | Anti-tacking agent |
| Glyceryl monostearate | 3.000 | mg | Anti-tacking agent |
| Ethanol | 664.286 | mg | Organic solvent |
| Acetone | 664.286 | mg | Organic solvent |

Method of Preparation of Enteric Coating

Hydroxypropyl methylcellulose phthalate HP-55 and dibutyl sebacate are dissolved in the mixture of ethanol and acetone. Talcum and glyceryl monostearate are suspended in obtained solution. The dispersion is bottom sprayed in fluid-bed device onto the core material covered with subcoating.

TABLETS (total weight 800 mg)

| | | | |
|---|---|---|---|
| Core material covered with | 400.000 | mg | |
| subcoating and enteric coating | | | |
| Lactose | 200.000 | mg | Tablet filler |
| Microcrystalline cellulose | 188.000 | mg | Tablet filler |
| Crosscarmelose sodium | 11.000 | mg | Disintegrating agent |
| Magnesium stearate | 1.000 | mg | Glidant |

Method of Preparation of Tablets

Core material covered with subcoating and enteric coating is mixed with lactose, microcrystalline cellulose, crosscarmelose sodium and magnesium stearate and compacted into tablets.

TABLET COATING (2.5% application,
coating weight 20.000 mg)

| | | |
|---|---|---|
| Hydroxypropylcellulose | 12.800 | Film forming polymer |
| Triethyl citrate | 1.000 | Plasticizer |
| Polysorbate | 0.600 | Surface active agent |
| Titan dioxide | 4.120 | |
| Pigment red | 0.200 | |
| Pigment yellow | 0.080 | |
| Talcum | 1.200 | Anti-tacking agent |
| Ethanol | 200.000 | Organic solvent |

Method of Preparation of Tablet Coating

Hydroxypropylcellulose, dibutyl sebacate and polysorbate are dissolved in ethanol. Titan dioxide, pigments and talcum are disperses in obtained solution. Obtained coating dispersion is sprayed onto tablets in coating pan.

### EXAMPLE 7

### Comparison of Stability of Non-Formulated Active Substance and the Stable Pharmaceutical Composition According to the Present Invention, Comprising the Active Substance in the Form of Solid Solution

Comparison of the total amount of degradation products and related substances in non-formulated active substance and in the stable pharmaceutical composition according to the present invention comprising the active substance in the form of solid solution is presented by the graph 1. The pharmaceutical composition is prepared as described in example 2. The amount of degradation products and related substances is determined by HPLC method.

[image]

The results obtained after 14 days storage on 40° C. show that relative to the starting analysis the increase of the amount of degradation products and related substances in the active substance esomeprazole magnesium is three times higher that the increase in the amount of of degradation products and related substances in the stable pharmaceutical composition comprising the active substance esomeprazole magnesium in the form of solid solution.

The results obtained after 6 days storage on 60° C. show even more significant difference in the increase of the amount degradation products and related substances. In the case of non-formulated active substance esomeprazole magnesium the increase of degradation products and related substances stored under said storage conditions, relative to the starting analysis, is four times higher than the increase in the case of the stable pharmaceutical composition comprising the active substance esomeprazole magnesium in the form of solid solution, stored under the same storage conditions.

### EXAMPLE 8

### Comparison of Stability of Non-Formulated Active Substance and the Stable Pharmaceutical Composition According to the Present Invention, Comprising the Active Substance in the Form of Solid Solution

Comparison of the total amount of degradation products and related substances in non-formulated active substance and in the stable pharmaceutical composition according to the present invention comprising the active substance in the form of solid solution is presented in graph 2. The pharmaceutical composition is prepared as described in example 5. The amount of degradation products and related substances is determined by HPLC method.

[image]

[image]

The results obtained after 6 days storage on 60° C. show significant difference in the increase in the amount of degradation products and related substances. In the case of non-formulated active substance esomeprazole magnesium the increase of degradation products and related substances stored 6 days on 60° C., relative to the starting analysis, is more then 10 times higher than in the case of the stable pharmaceutical composition comprising the active substance esomeprazole magnesium in the form of solid solution, stored under the same storage conditions.

### CONCLUSION

The comparative stability test results disclosed in examples 7 and 8 undoubtedly demonstrate significant improvement of stability of the active substance esomeprazole magnesium by being transformed into solid solution in the stable pharmaceutical composition according to the present invention, in comparison to non-formulated active substance esomeprazole magnesium.

1. A stable pharmaceutical composition comprising an active substance that is unstable in acidic medium, unstable when stored in the presence of water and at the same time sensitive to heating, wherein said active substance is stabilized by being transformed into the form of a solid solution.

2. The stable pharmaceutical composition according to claim 1, wherein said stable pharmaceutical composition comprises:
a) a core material comprising an inert core and a solid solution layer,
b) optionally one or more subcoatings, and
c) an enteric coating.

3. The stable pharmaceutical composition according to claim 2, wherein the inert core is a non-pareil bead, a crystal, a granule, a pellet, a spherule, a micro tablet or a tablet.

4. The stable pharmaceutical composition according to claim 3 wherein the non-pareil bead is made of microcrystalline cellulose, sucrose, starch or any combination thereof.

5. The stable pharmaceutical composition according to claim 2, wherein the solid solution layer comprises a solid solution of an active substance in a polymer carrier and pharmaceutical excipients for film coating.

6. The stable pharmaceutical composition according to claim 2, wherein the active substance is a benzimidazole derivative selected from the group consisting of omeprazole, lansoprazole, timoprazole, rabeprazole, pantoprazole, leminoprazole, pariprazole, a pharmaceutically acceptable salt thereof, an enantiomer thereof and a pharmaceutically acceptable salt of an enantiomer thereof.

7. The stable pharmaceutical composition according to claim 1, wherein the active substance is esomeprazole or a pharmaceutically acceptable salt thereof.

8. The stable pharmaceutical composition according to claim 1, wherein the active substance is a magnesium salt of esomeprazole.

9. The stable pharmaceutical composition according to claim 5, wherein the polymer carrier is selected from the group consisting of a polyvinylpyrrolidone, a cellulose derivative, a polymethacrylate and a polyethylene glycol and any combination thereof, in particular a polyvinylpyrrolidone.

10. The stable pharmaceutical composition according to claim 5, wherein the weight ratio between the active substance and the polymer carrier present in the core is from 1:1 to 1:6, preferably from 1:1 to 1:3.

11. The stable pharmaceutical composition according to claim 5, wherein the pharmaceutical excipients for film coating are selected from the group consisting of one or more plasticizing agents, one or more surface active agents and one or more anti-tacking agents.

12. The stable pharmaceutical composition according to claim 2, wherein the subcoating comprises at least one film forming polymer.

13. The stable pharmaceutical composition according to claim 12 wherein the film forming polymer is selected from the group consisting of a cellulose ether, a polyvinylpyrrolidone, a vinyl pyrrolidone/vinyl acetate copolymer and a polymethacrylates, and combinations thereof,

14. The stable pharmaceutical composition according to claim 13 wherein the film forming polymer is a cellulose ether selected from the group consisting of a hydroxypropyl methylcellulose, a hydroxypropylcellulose, a methylcellulose, a sodium carboxymethylcellulose, and an ethylcellulose.

15. The stable pharmaceutical composition according to claim 2, wherein the enteric coating comprises at least one polymer selected from the group consisting of a copolymer of methacrylic acid, an ethyl cellulose, shellac, an ester of a hydroxyalkylcellulose, one or more plasticizers, selected from the group consisting of a polyethylene glycol, cetyl alcohol, an olive oil, a castor oil, a monoglyceride, diethyl phthalate, triethyl citrate and dibutyl sebacate, in particular dibutyl sebacate.

16. A pharmaceutical dosage form comprising a sachette or a capsule comprising a stable pharmaceutical composition according to claim 2.

17. A method of stabilization of an active substance that is unstable in acidic medium, unstable when stored in the presence of water and at the same time sensitive to heating, in the pharmaceutical composition, which comprises transforming said active substance into the form of a solid solution.

18. The method of stabilization of the active substance according to claim 17 wherein the pharmaceutical composition is the stable pharmaceutical composition defined by claim 17.

19. A process for the preparation of the stable pharmaceutical composition according to claims 1, characterized in that it comprises the following steps:
a) providing a core material comprising preparing and applying of a solid solution layer on the surface of the inert core,
b) optionally applying one or more subcoatings on the core material,
c) applying enteric coating.

20. The process according to claim 19 wherein the preparation of the solid solution comprises the following steps:
a) dissolving the active substance and the polymer carrier in one or more organic solvents selected from the group consisting of ethanol and acetone,
b) dissolving or dispersing the pharmaceutical excipients for film coating in the obtained solution,
c) spraying the obtained dispersion onto the surface of inert cores, followed by simultaneous solvent evaporation and forming of the solid solution layer on the surface of inert cores.
